(19) United States
(12) Patent Application Publication
(10) Pub. No.: US 2009/0024007 A1
(43) Pub. Date: Jan. 22, 2009

(54) SYSTEM TO TRANSMIT VITAL SIGNALS FROM MOVING BODY WITH DYNAMIC EXTERNAL DISTURBANCE AND TO COMPENSATE ARTIFACT THEREOF

(75) Inventors: Kyoung-Joung Lee, Gangwon-do (KR); Ho-Dong Park, Gangwon-do (KR); Mi-Hye Song, Gyeongsangbuk (KR)

Correspondence Address: FISH & RICHARDSON P.C., P.O. BOX 1022, MINNEAPOLIS, MN, 55440-1022, US

(73) Assignee: Industry-Academic Cooperation Foundation, Yonsei University, Seoul (KR)

(21) Appl. No.: 11/996,466

(22) PCT Filed: Jul. 29, 2005
(86) PCT No.: PCT/KR2005/002489

§ 371 Date: Jul. 23, 2008

(30) Foreign Application Priority Data
Jul. 28, 2005 (KR) 10-2005-0068693

(51) Int. Cl. A61B 5/00

(52) U.S. Cl. 600/301

(57) ABSTRACT

The present invention relates to a system to transmit vital signals from a moving human body in the presence of dynamic external disturbance and to compensate for artifacts thereof. More specifically, it relates to a system to compensate for artifacts caused by external noise during measurement of vital signals, and to display index of vibration on the monitoring system through interpretation of the vibration signals which affect the physiological state of the body and the measurement of vital signals, due to external disturbance factors, when noise from external disturbances such as vibration of a moving ambulance vehicle, vibration of a remote transmitting diagnostic device attached to the patient, or movement of the patient, is mixed into the transmitted signals. The present invention provides a start code, a data code, a degree of vibration and movement level, a three axis acceleration value and signals of three axis acceleration and measured vital signals. Thus it can improve the accuracy of measurement of vital signals (ECG, NiBP, SPO2, TEMPERATURE, Respiration) under adverse conditions with much vibration such as in moving vehicles. Furthermore, as the present invention can be attached to conventional vital signals measurement modules on the patient through a simple interface part, it can minimize inaccuracy due to vibration and dynamic disturbance in measurement of vital signals during transport of the patient by vehicle or otherwise. Furthermore, the present invention can compensate for artifacts caused by external noise during measurement of vital signals, and display index of vibration on the monitoring system through interpretation of the vibration signals which affect the physiological state of the body and the measurement of vital signals, due to external disturbance factors, when noise from external disturbances such as vibration of a moving ambulance vehicle, vibration of a remote transmitting diagnostic device attached to the patient, or movement of the patient, is mixed into the transmitted signals.

### TECHNICAL FIELD

The present invention relates to a system to transmit vital signals from a moving human body in the presence of dynamic external disturbance and to compensate for artifacts thereof. More specifically, it relates to a system to compensate for artifacts caused by external noise during measurement of vital signals, and to display index of vibration on the monitoring system through interpretation of the vibration signals which affect the physiological state of the body and the measurement of vital signals, due to external disturbance factors, when noise from external disturbances such as vibration of a moving ambulance vehicle, vibration of a remote transmitting diagnostic device attached to the patient, or movement of the patient, is mixed into the transmitted signals.

### BACKGROUND ART

Medical devices for measurement of vital signals generated in the body, such as EEG, EKG, electrooculogram, blood pressure and EMG are used to diagnose, to measure and to store vital signals for use in medical examination and treatment.

Conventional vital signals measurement medical apparatus performs all the functions of measuring, storing, opening and reading vital signals in one independent apparatus. In such a case, to measure, open, and read the vital signals, the independent vital signals medical apparatus must be located near the patient in the presence of a medical professional.

To overcome these limits, there have been attempts to make possible opening and reading of vital signal measurement apparatus situated outside the examination room by separating vital signals measurement function from the opening and reading functions and connect the separated function with communication means.

Furthermore, as medical technology advances, the technology of transmitting vital signals a long distance is developing and now the method of measuring EKG in an ambulance or the home for transmission to a medical center is widely used.

ECG monitoring apparatus can be used to diagnose diseases and conditions associated with the heart, include Holter ECG, resting ECG and stress ECG monitors.

A prior art of this invention, KOREA Patent laid open publication No. 10-2005-0042964 described in FIG. 1, show compound vital signal measurement device and its system, that connects the compound vital signals measurement device and personal information terminal with a personal computer, which comprises a short distance wireless communication part for wireless communication with external devices that are a short distance away, a multiple measurement module interface that can connect to measurement modules, means for measuring each of the various vital signals, multiple measurement modules having removable connections with the measurement module interface, and a controller part which transmits measurement instructions to a specified measurement module through the measurement module interface according to measurement instructions received by the short distance wireless communication part, and which transmits through the short distance wireless communication part the measurement results data of each measurement module received through the measurement module interface.

However, this measurement system requires a static or unmoving condition of the human body, so it is impossible to measure the signals, if the human body is moving and noise from external disturbance is mixed into the measurement signals.

KOREA Patent laid open publication No. 10-1997-0014722 relates to a portable vital signal monitoring system as shown in FIG. 2, which comprises a vital signals holter apparatus which attaches to a patient body to measure vital signals data, and transmits through a wireless communication network to a vital signals monitoring center whenever an abnormal condition is detected by abnormality in the measured vital signals data, and a vital signals monitor server apparatus which transmits message to the vital signals holter apparatus when vital signals data reflecting an abnormal condition is received through the wireless communication network from the vital signals holter apparatus. Although this technology can measure remote vital signals, it also has the fatal fault that it cannot handle noise such as that from external disturbance from a moving body.

KOREA Patent laid open publication No. 10-2001-0103920 relates to a remote medical apparatus, which provide a testing means to generate vital signals data by measuring the vital signals of patients, a reading means to generate the read data by reading the vital signals, an opening means to open the vital signals and the read data, a data storage and management means to transmit the requested data when the reading and opening means request the stored vital signals data and the stored read data, after receiving and storing the vital signals data and the read data from the testing means and reading means. This technology also monitors patients remotely, but is very sensitive to external disturbance such as vibration of a patient that is moving by ambulance or on foot, and has restricted use for remote diagnosis due to the necessary requirement that the patient must not be moving.

Accordingly, there is a demand for a vital signals measurement medical apparatus which can obtain accurate measurement data by removing the influence of external disturbance factors, when the signals transmitted include noises caused by external factors such as external vibration and a moving body.

### DISCLOSURE OF INVENTION

### Technical Problem

The present invention provides a system to display a vibration index on a monitoring system through interpreting vibration signals due to external disturbance factors which influence the physiological state and vital signals measurement and to compensate for artifacts caused by external disturbance, when noise caused by external disturbance is mixed in with transmitted vital signals.

The present invention also provides a method to display a vibration index on a monitoring system through interpreting vibration signals due to external disturbance factors which influence the physiological state and vital signals measurement and to compensate for artifacts caused by external disturbance, when noise caused by external disturbance is mixed in with transmitted vital signals.

### Technical Solution

According to the present invention, the system of transmitting vital signals from a moving body in the presence of dynamic external disturbance and compensating for artifacts thereof, comprises a three axis acceleration sensors part which detects acceleration in each of the directions of x, y, and z axes, and converts them to electrical signals, a pre-amplifier part amplifying the output of the three axis acceleration sensors and providing only the band signals for digital signal processing by removing high frequency noise, an A/D converter part converting the output signal of the pre-amplifier into a digital signal, a digital signal processing part which determines stopping and moving states during walking or vehicle transport of a person by calculating the three axis acceleration signals output from the A/D converter, and a transmitter transmitting the results of the digital processing part and the three axis acceleration signals. The digital signal processing part discriminates walking, stopping during walking, stopping during vehicle transport, and moving vehicle states. The transmitter transmits vital measurement signals together with digital signal processing results and three axis acceleration signals.

Furthermore, the digital signal processing part comprises a memory part storing and receiving X, Y, and Z axis acceleration data from the A/D converter set by time frame units, a differential calculation part differentiating on the basis of a zero level of data signals by reading the X, Y, and Z axis acceleration data stored in the memory part by set time frame units, a mean value calculation part averaging the X, Y, and Z axis acceleration differentiation data which is the output signal of the differential calculation part, Z axis mean value comparator part which determines whether a mean value of Z axis acceleration differential data obtained from the mean value calculation part is larger than the first reference value, a X, Y mean value comparator part which, if mean value of Z axis acceleration differential data at the Z axis mean value comparator part is less than or equal to a certain reference value, determines whether the mean value of X, Y axis acceleration differential data obtained from the mean value calculation part is larger than a second reference value, determines that the vehicle has stopped if it is larger than the second reference value and determines that a person has stopped walking if it is equal to or smaller than the second reference value, a frequency analysis part obtaining a mean spectrum of the Z axis acceleration differential data by taking an FFT of the output signals of the differential operation part, a Z axis spectrum comparator part which determines that a person is walking if it is larger than a third reference value, and determines that a vehicle is moving if it is equal to or smaller than the third reference value by determining whether the Z axis mean spectrum obtained from the frequency analysis part is larger than the third reference value.

Another embodiment of the present invention provides a method to transmit vital signals from a moving body in the presence of dynamic external disturbance and to compensate for artifacts thereof, which first converts the output signals obtained from the three acceleration sensors into digital signals through the A/D converter. After they are converted into digital signals, acceleration differential signals for each of the X, Y, and Z axial directions are calculated from the output three axis acceleration signals. The mean value of the differentiated acceleration signals is calculated to obtain the mean values from the differential operation output signals. It is determined that the human body is moving if the mean value of the acceleration differential signals of Z axis direction among the mean values of the acceleration differential signals for the X, Y, Z axis direction is greater than the first reference value, and it is determined that the human body is not in motion if it is less than or equal to the first reference value. When it is determined that the human body is not in motion as the mean value of Z axis acceleration differential signals is less than or equal to the first reference value, it is determined that the vehicle is not in motion during transport of the body when the mean value of X, Y axis acceleration differential signals is compared with the second reference value and is determined to be greater, and it is determined that the body is not in motion during walking if the mean value of X,Y axis acceleration differential signals is smaller than the second reference value. The Z axis mean spectrum is calculated when it is determined that the body is in motion as the mean value of Z axis acceleration differential signals is greater than the first reference value. The Z axis mean spectrum value is compared with the third reference value, and if the Z axis mean spectrum value is greater than the third reference value, then the body is determined to be moving on foot, and if the Z axis mean spectrum is smaller than the third reference value then the body is determined to be on a motion vehicle.

### ADVANTAGEOUS EFFECTS

The present invention provides a start code, a data code, a degree of vibration and movement level, a three axis acceleration value and a signals of three axis acceleration sensor part and the measured vital signals, so it can improve the accuracy of measurement of vital signals (ECG, NiBP, SPO₂, TEMPERATURE, Respiration) under adverse conditions with much vibration such as in moving vehicles. Furthermore, as it is possible to attach a simple interface part to the patient with conventional vital signals measurement modules, the present invention can minimize inaccuracy due to vibration and dynamic disturbance in the measurement of vital signals during transport of the patient in an ambulance or otherwise.

Furthermore, the present invention provides improved accuracy of the measurement module and minimized influence to measurement by taking into account the influence of noise at the place of measurement, by attaching at any place of measurement to receive vibration signals and motion noise and communicating the vital signals measurement module through RF communication and to compensate for motion noise.

Even though noise caused by external disturbance such as the interior vibration of a moving ambulance, or the vibration of the patient on which a remote diagnostic device is attached, the present invention has the effect of compensating artifacts caused by external noise in the measurement of vital signals by interpreting the vibration signals caused by external disturbance which influence the physiological state of the body and measurement of vital signals and displaying the vibration index on the monitoring system.

### BRIEF DESCRIPTION OF THE DRAWINGS

FIG. 1 is an illustration of a conventional vital signals measurement apparatus.

FIG. 2 is another illustration of a conventional vital signals measurement apparatus.

FIG. 3 is a diagram of a system to transmit vital signals from a moving body in the presence of dynamic external disturbance and to compensate for artifacts thereof, according to an embodiment of the present invention.

FIG. 4 shows output signals from a human body moving on foot from a three axis acceleration sensor part of FIG. 3, according to an embodiment of the present invention.

FIG. 5 shows output signals from a human body during vehicle transport, from a three axis acceleration sensor part of FIG. 3, according to an embodiment of the present invention.

FIG. 6 shows differential signals from output signals of a human body moving on foot from a digital signals processing part of FIG. 3, according to an embodiment of the present invention.

FIG. 7 shows differential signals from output signals of a human body during vehicle transport, from a digital signals processing part of FIG. 3, according to an embodiment of the present invention.

FIG. 8 shows mean graphs for the three axis acceleration signals of a human body moving on foot and during vehicle transport, from a digital signals processing part of FIG. 3, according to an embodiment of the present invention.

FIG. 9 shows the result of FFT analysis for the three axis acceleration signals of a human body moving on foot and during vehicle transport, from a digital signals processing part of FIG. 3, according to an embodiment of the present invention.

FIG. 10 is a diagram illustrating the digital signals processing part of FIG. 3.

FIG. 11 is a diagram illustrating information received from a transmitter part of FIG. 3.

FIG. 12 is a flow diagram of the three axis acceleration data processing at the digital signals processing part of FIG. 3.

FIG. 13 is a data packet structure received from a transmitter part of FIG. 3 according to an embodiment of the present invention.

FIG. 14 is a diagram illustrating a method of removing vibration signals included in vital signals, according to an embodiment of the present invention.

FIG. 15 is a model of adaptive filtering of FIG. 14, according to an embodiment of the present invention.

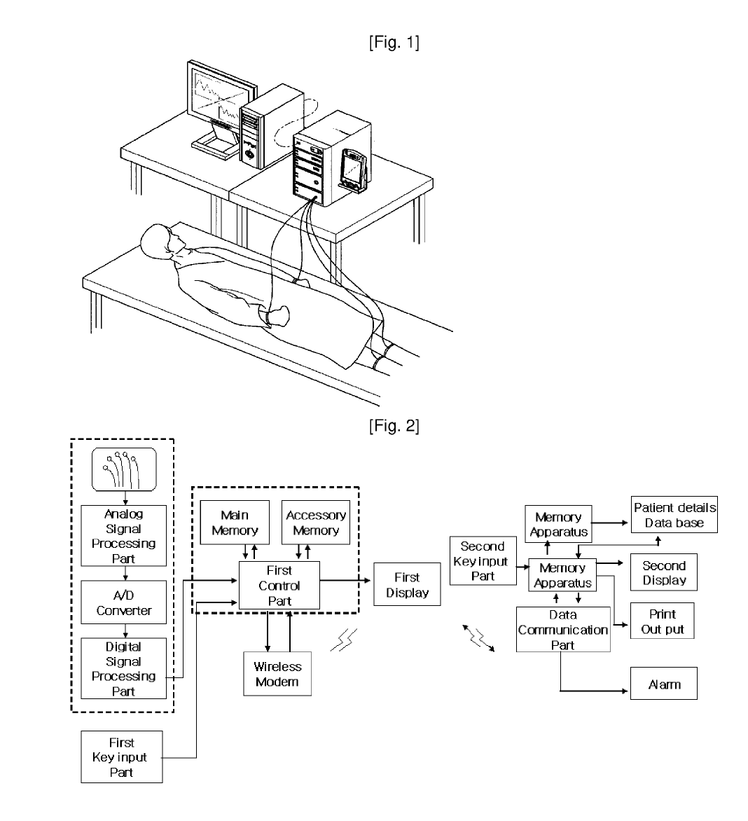

### BEST MODE FOR CARRYING OUT THE INVENTION

The features and advantages of the present invention and the methods to attain them will become more apparent by the following description of exemplary embodiments thereof with reference to the attached drawings. However, the invention is not limited to these embodiments, but can be realized in various forms. These embodiments are given for the purpose of illustration and scope of the present invention will be defined by the claims. Like reference numerals in the drawings denote like elements.

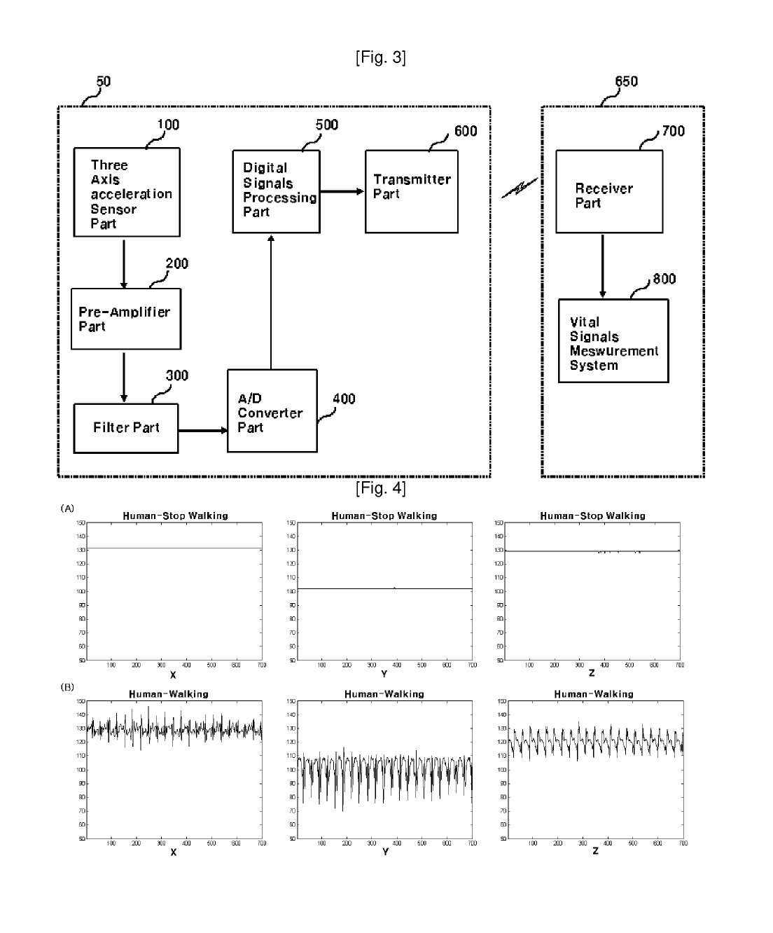

FIG. 3 shows a diagram of a system to transmit vital signals from a moving body in the presence of dynamic external disturbance and to compensate for artifacts thereof according to an embodiment of the present invention. According to FIG. 3, the system to transmit vital signals from a moving body in the presence of dynamic external disturbance and to compensate for artifacts thereof, comprise a three axis acceleration sensor part (100), a pre-amplifier part (200), a filter part (300), an A/D converter part (400), a digital signals processing part (500), a transmitter part (600), a receiver part (700), and a vital signals measurement system (800).

The three axis acceleration sensor part (100) is attached to an ambulance or a patient, converts the X, Y, Z axis acceleration signals into electric signals, and outputs it. The three axis acceleration sensor part (100) separately measures the X, Y, Z axis acceleration from a three dimensional vibration.

The pre-amplifier part (200) amplifies the output from the three axis acceleration sensor part (100) and transmits it to the filter part (300). First, the filter part (300) carries out analog filtering to remove 60 Hz noise and high frequency noise in order to supply only signals in the vital signals band. The signals pre-processing part is composed of the pre-amplifier part (200) and the filter part (300). The A/D converter part (400) converts the analog filtered signals output from the filter part (300) into digital signals.

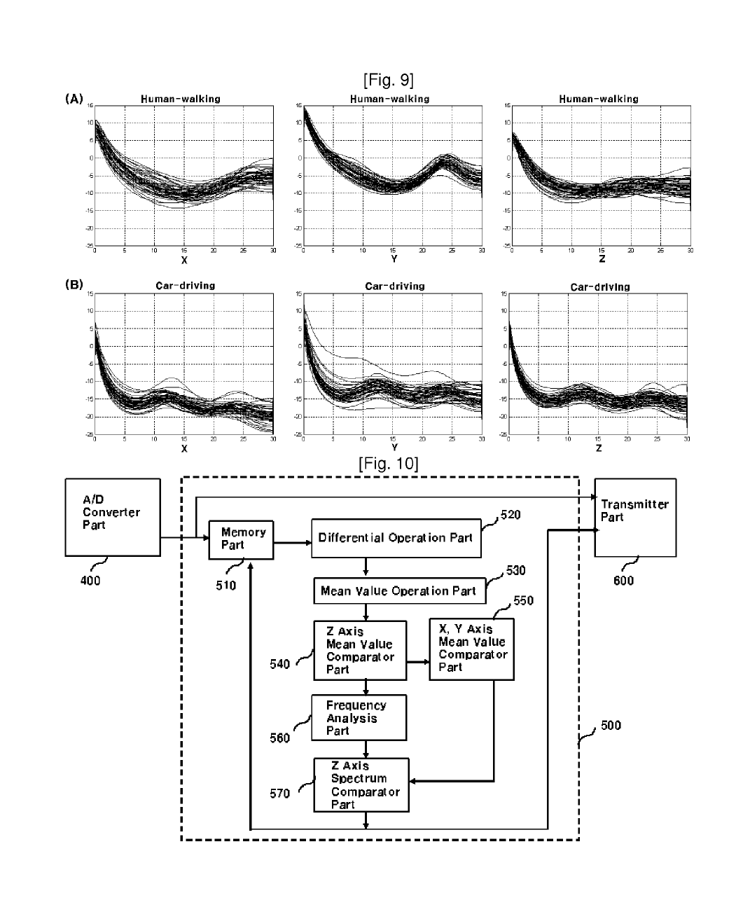

The digital signals processing part (500) detects the vibration signals and movement level by calculating the output signals from the A/D converter part (400). The digital signals processing part (500), as described in FIG. 9, contain a memory part (510), a differential operation part (520), a mean value operation part (530), Z axis mean value comparator part (540), X,Y axis mean value comparator part (550), a frequency analysis part (560), and a Z axis spectrum comparator part (570). The digital signals processing part (500) determines whether a body is moving on foot, has stopped while moving on foot, is moving by vehicle transport, or has stopped during vehicle transport, transmits the states determination data to the transmitter part (600), and also transmits acceleration data received from the A/D converter part (400) to the transmitter part (600).

The transmitter part (600) transmits the output signals from the digital signals processing part (500) through wireless communication. The receiver part (700) transmits the signals received from the transmitter part (600) to the vital signals measurement system (800). The vital signals measurement system (800) extracts accurate vital measurement signals by removing vibration signals from the vital measurement signals received from the receiver part.

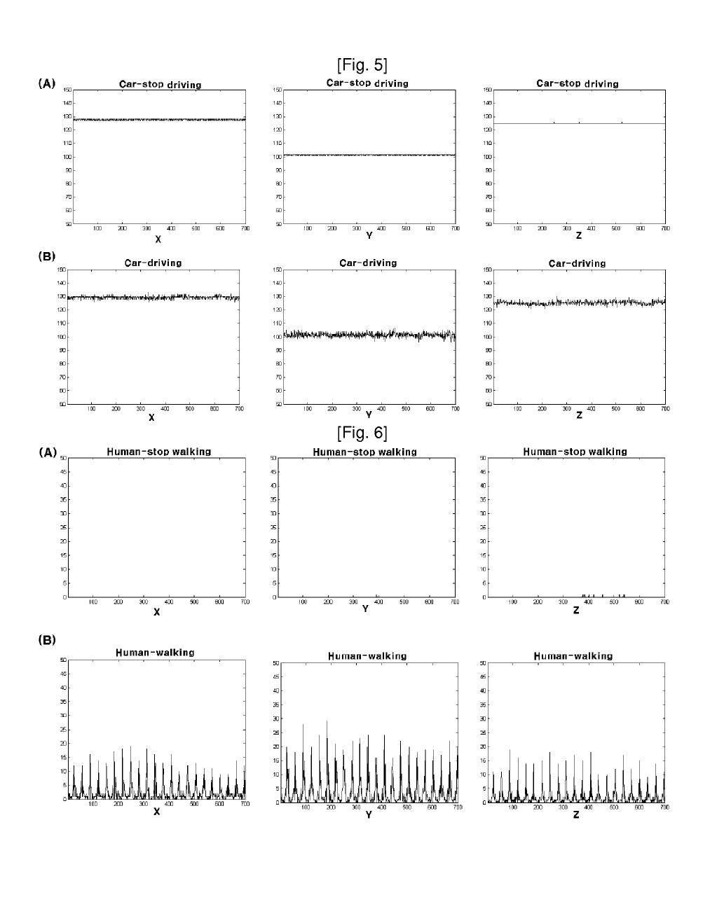

FIG. 4 shows output signals from a human body moving on foot from the three axis acceleration sensor part of FIG. 3, according to an embodiment of the present invention. FIG. 5 shows output signals from a human body during vehicle transport from car moving at a three axis acceleration sensor part of FIG. 3, according to an embodiment of the present invention. FIG. 4(A) shows output signals in the X, Y, Z axis direction from the three axis acceleration sensor part (100) attached to a patient who has stopped moving. FIG. 4(B) shows output signals in the X, Y, Z axis direction from the three axis acceleration sensor part (100) attached to a patient who is moving on foot.

FIG. 5(A) shows a output signals in the X, Y, Z axis direction from the three axis acceleration sensor part (100) attached to a patient being transported by a vehicle which has stopped moving. FIG. 5(B) shows output signals in the X, Y, Z axis direction from the three axis acceleration sensor part (100) attached to a patient being transported by a moving vehicle.

In FIG. 4 and FIG. 5, the amplitude of signals is smaller in the non-moving states (FIG. 4(A) and FIG. 5(A)), and is greater in the moving states by foot or by vehicle transport (FIG. 4(B) and FIG. 5(B)). Furthermore, the amplitude of signals is relatively greater in the non-moving state during vehicle transport (FIG. 5(A)) compared to that of the non-moving state patient on foot (FIG. 4(A)). This is because the amplitude of output signals include the vehicle's engine vibration. Furthermore, the amplitude of signals is relatively greater during movement on foot (FIG. 4(B)) compared to that during movement on a vehicle (FIG. 5(B)). The amplitude will be relatively greater in case of movement on foot because there is more movement of the patient on foot than during vehicle transport.

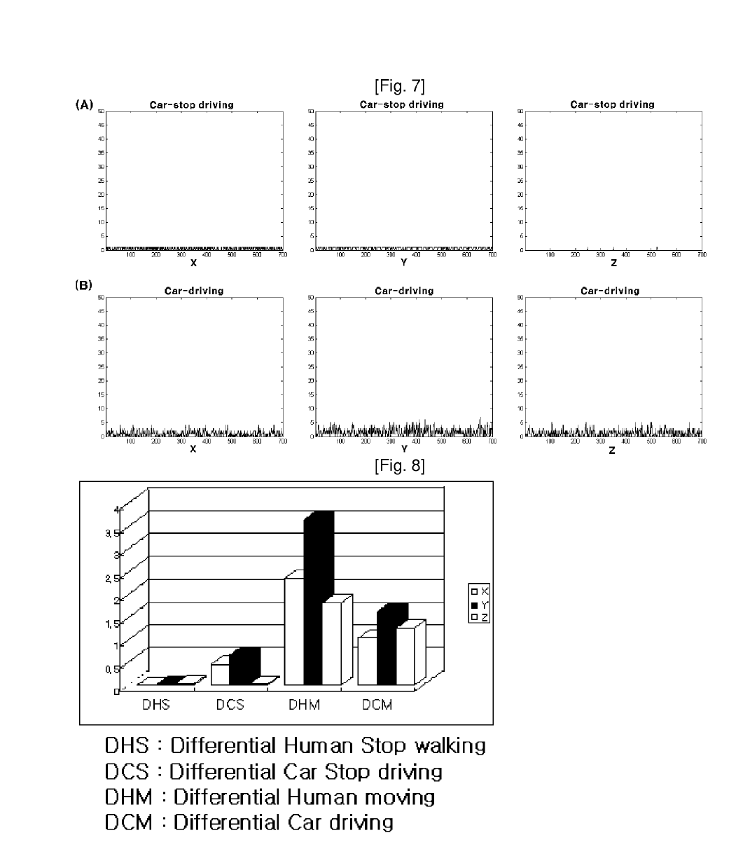

FIG. 6 shows differential signals from output signals of a walking patient, at a digital signals processing part of FIG. 3 according to an embodiment of the present invention. FIG. 7 shows differential signals from output signals of a patient in a moving vehicle at a digital signals processing part of FIG. 3 according to an embodiment of the present invention. That is, FIG. 6 shows differential signals that is a differentiated value of each of the signals of FIG. 4, and FIG. 7 shows differential signals that is a differentiated value of each of the signals of FIG. 5.

FIG. 6 and FIG. 7 shows the amplitude magnitude of each of signals without a baseline drift, since it was differentiated in such a way as to subtract the subsequent sample signal from the previous sampled signal. Consequently, the signal amplitudes in the case non-movement (FIG. 6(A) and FIG. 7(A)) are substantially smaller than in the case of a patient moving on foot or on a vehicle (FIG. 6(B) and FIG. 7(B)). The amplitude of signals in the case of a patient in a stopped vehicle (FIG. 7(A)) is comparatively larger than in the case of a patient who has stopped walking (FIG. 6(A)), and the amplitude of signals in the case of a patient who is walking (FIG. 6(B)) is comparatively larger than in the case of a patient on a moving vehicle (FIG. 7(B)). That is, under moving conditions higher signal levels are detected for a patient who is walking than for a patient on a moving vehicle.

FIG. 8 is a mean graph for the three axis acceleration signals in the case of a patient moving on foot or on a vehicle, at a digital signals processing part of FIG. 3 according to an embodiment of the present invention. That is, FIG. 8 shows the distribution of mean values of signals within a set time period, from three axis acceleration sensors part (100) output in the X, Y, Z directions for non-moving and moving conditions for a patient moving on foot or on a vehicle. The DHS, described in FIG. 8, shows mean values over a set time period of differential signals from a patient who has stopped walking as in FIG. 6(A) i,e, shows mean values of Differential Human Stop Walking, and the DCS shows mean values over a set time period of differential signals from a patient who is on a stopped vehicle FIG. 7(A) i,e, shows a mean of Differential Car Stop Driving, and the DHM shows mean values over a set time period of differential signals from a patient walking as in FIG. 6(B) i,e, shows a mean of Differential Human Moving, and the DCM shows mean values over a set time period of differential signals from a patient on a moving vehicle as in FIG. 7(B) i,e, shows a mean of Differential Car Driving.

Output Z axis signals allow discrimination of whether the condition is a stopped or moving condition, and the output X, Y axis signals allow discrimination of whether the stopped state is during vehicle transport or walking. As human movement generally outputs large signals during walking, in reality, it is difficult to maintain the accuracy of direction coordinates the signals of three axis acceleration sensor (100) during human movement, so it is often difficult to judge whether the transport is on foot or on a vehicle with a simple magnitude distribution. Thus the present invention analyzes the spectrum of the output signals of three axis acceleration sensor (100) during movement.

FIG. 9 is an analysis result of FFT for the three axis acceleration signals from a patient moving on foot and on a vehicle at a digital signals processing part according to an embodiment of the present invention.

That is, FIG. 9 shows the spectrum of differential signals of X, Y, Z axis directions during movement on foot and an a vehicle FIG. 9(A) shows the spectrum of differential signals of X, Y, Z axis directions of three axis acceleration sensor (100) during movement on foot, and (FIG. 9(B)) shows the spectrum of differential signals of X, Y, Z axis directions of three axis acceleration sensor (100) during movement on a vehicle.

In FIG. 9, for signals in the approximately 5 Hz frequency or higher, the spectrum distribution is at a higher level during movement on foot than during movement on a vehicle. Thus, this characteristic of signals of three axis acceleration sensor (100) can be used to discriminate between stopped or moving states during transport on foot or on a vehicle.

FIG. 10 is a diagram illustrating the digital signals processing part of FIG. 3. In FIG. 10, the digital signals processing part provides a memory part (510), a differential operation part (520), a mean value operation part (530), Z axis mean value comparator part (540), X,Y axis mean value comparator part (550), a frequency analysis part (560), and Z axis spectrum comparator part (570).

The memory part (510) stores the X, Y, Z axis acceleration data received from the A/D converter (400) in set time frame units. The differential operation part (520) reads the X, Y, Z axis acceleration data stored in the memory part (510) in set time frame units, and differentiates it. The mean value operation part (530) takes a mean of the X, Y, Z axis acceleration differential data output from the differential operation part (520). The Z axis mean value comparator part (540) determines whether the mean value of the Z axis acceleration differential data is greater than the first reference value. The X, Y axis mean value comparator part (550), if the mean value of the Z axis acceleration differential data obtained from the Z axis mean value comparator part (540) is equal to or smaller than a certain reference value, determines whether the mean value of the acceleration differential signals of X, Y axis direction obtained from the mean value operation part (530) is greater than the second reference value, and if it is greater, it is determined to be a stopped condition during vehicle transport, and if equal to or smaller, then a stopped condition during walking. The frequency analysis part (560) obtains the mean spectrum of the Z axis. That is, the frequency analysis part (560) obtains the mean spectrum of Z axis acceleration differential data by obtaining FFT.

The Z axis spectrum comparator part (570) compares whether the mean value of the acceleration differential signals of X, Y axis direction obtained from the mean value operation part (530) is greater than the third reference value and determines that the patient is moving on foot if it is greater, and that the patient is moving on a vehicle if it is equal to or smaller.

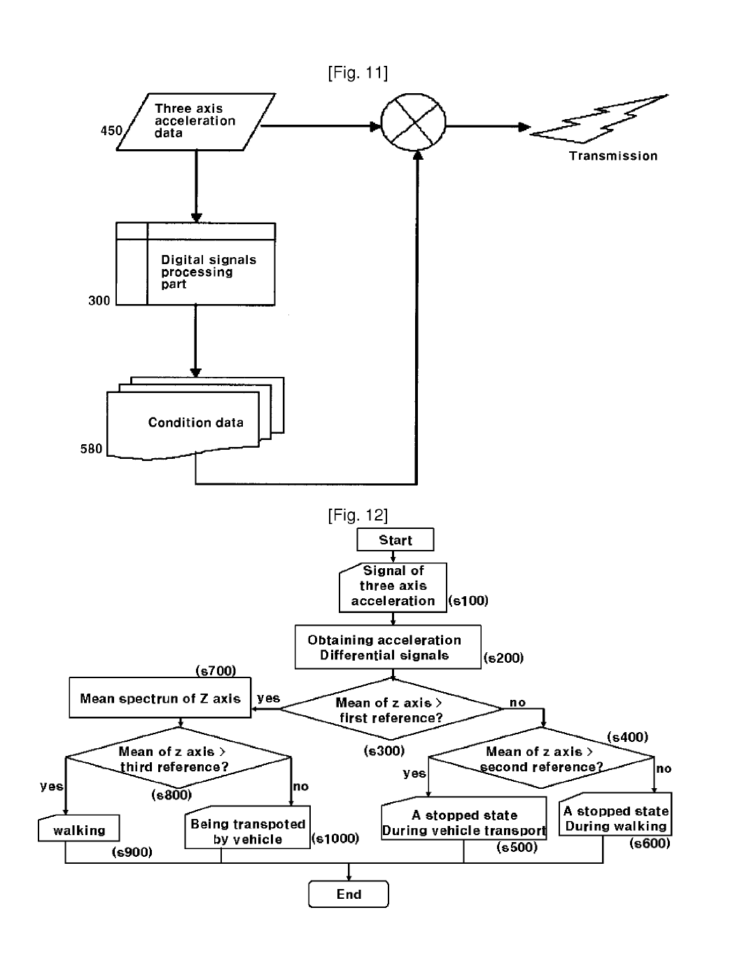

FIG. 11 is a diagram illustrating information received from a transmitter part of FIG. 3. It transmits the condition data (580) output from the digital signals processing part (500) which discriminates between stopped and moving states during walking or vehicle transport, together with the three axis acceleration data (450) received through the A/D converter (400) from the three axis acceleration sensor part (100). Although not shown in FIG. 11, the present invention can provide transmission of various measured vital signals together with the three axis acceleration data (450) and the condition data (580).

FIG. 12 is a flow diagram for the three axis acceleration data processing at the digital signals processing part of FIG. 3.

The digital signals processing part (500) receives signals of the three axis acceleration output from the A/D converter (100) (s100), calculates the acceleration differential signals of X, Y, Z axis directions (s200), and obtains the mean from the differential signals. From this, the mean value of the acceleration differential signals of Z axis direction is compared with the first reference value, and it is determined to be a moving state if the mean value of the acceleration differential signals of Z axis direction is greater than the first reference value, and determined to be a stopped state if it is smaller than the first reference value (s300).

If the mean value of the acceleration differential signals of Z axis direction is smaller than the first reference value and it is determined to be a stopped state, then the mean value of the acceleration differential signals of X,Y axis direction is compared (s400) with the second reference value, and when the mean value of the acceleration differential signals of X,Y axis directions is greater than the second reference value it is determined to be a stopped state during vehicle transport (s500), and when the mean value of the acceleration differential signals of X,Y axis direction is smaller than the second reference value it is determined to be a stopped state during walking (s600).

If it is determined to be a moving condition as the mean value of the acceleration differential signals of Z axis direction is greater than the first reference value, the mean spectrum of Z axis is calculated (s700), and the calculated mean spectrum of Z axis is compared with the third reference value (s800). If the mean spectrum of Z axis is greater than the third reference value the patient is determined to be walking (s900), and if the mean spectrum of Z axis is smaller than the third reference value the patient is determined to be transported by vehicle (s1000).

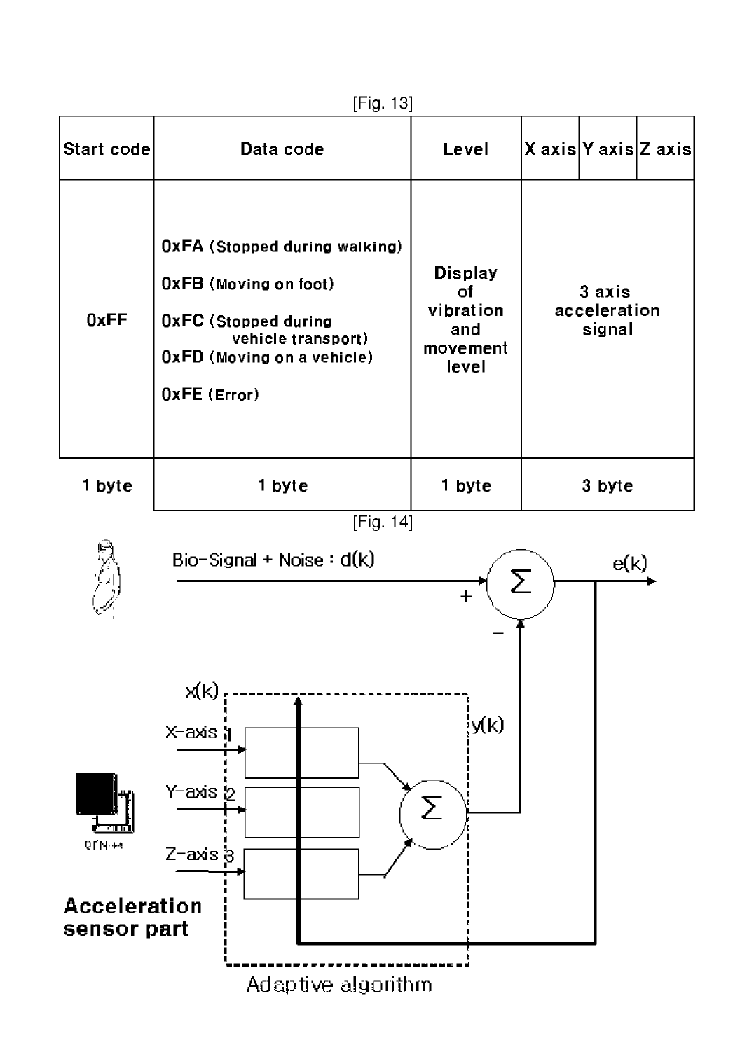

As it can be seen from the example in FIG. 13, the result determined by the above process is transmitted in a packet structure as a condition code.

FIG. 13 is an embodiment of packet structure of data transmitted by the transmitter part in FIG. 3. The data packet starts off with the Start code (0xFF), and the next Data code following shows the current movement condition. For example, if the data code is 0xFA, it indicates a stopped state during walking, and if the data code is 0xFD, it indicates movement by vehicle transport. Next, the level of vibration and movement is indicated with a numerical value (from 0 to 10). Subsequently, the three axis acceleration value is transmitted in the order of X, Y, and Z. Consequently, the data packet sequence is the same as FIG. 12, and the total number of data transmitted is 4 bytes.

The packet structure of the present invention is one embodiment, so the packet structure which provides a start code, a data code, a level of vibration and movement, and a three axis acceleration value can be modified in various ways by those skilled in the art.

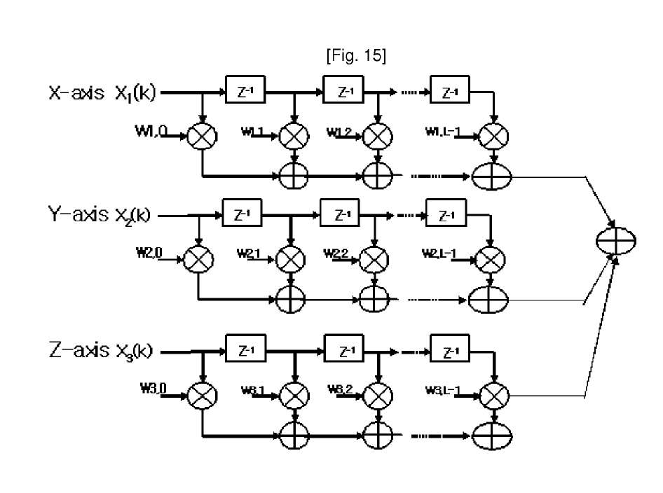

FIG. 14 is a diagram illustrating a method of removing vibration signals included in vital signals according to an embodiment of the present invention, and FIG. 15 is an example of a model of adaptive filtering of FIG. 14.

The measured vital signals (d(k)) contain noise (due to dynamic external disturbance). The acceleration signals (X(k): X₁(k), X₂(k), X₃(k)) measured at the three axis acceleration sensor is used as reference signals in order to remove the noise included in the measured vital signals. A weighted value (W₁(k)=[W_{1,1}(k), W_{1,2}(k), W_{1,L-1}(k) . . . ], W₂(k) [W_{2,1}(k), W_{2,2}(k), W_{2,L-1}(k), . . . ], W₃(k)=[W _{3,1}(k), W_{3,2}(k), W_{3,L-1}(k), . . . ]) is multiplied to reference signals and error is calculated according to Formula 1 by subtracting reference signals from the measured vital signals.

$$e ( k ) = d ( k ) - {\sum }_{i = 1}^{3} {W}_{i} ( k ) {X}_{i} ( k ) \qquad Formula 1$$

In Formula 1, d(k) is the measured vital signal, Wᵢ is the weighted value and Xᵢ is acceleration signals. Acceleration signals are of three axes so i is from 1 to 3.

The error is equivalent to the measured vital signals minus the weigh values multiplied by the acceleration signals. An adaptive algorithm compensates for the weight value until the error becomes the smallest, and the weight compensation formula is shown in Formula 2.

$$\begin{matrix}{W}_{i} ( k + 1 ) = {w}_{i} ( k ) + \frac{( 1 - β )}{{σ}^{2} {x}_{i} ( k )} e ( k ) {x}_{i} ( k ) & i = 1 , 2 , 3\end{matrix} \\ Here , \\ \frac{( 1 - β )}{{σ}^{2} {x}_{i} ( k )} \qquad Formula 2$$

is a constant showing step size as a μ value. Convergent velocity is determined from this value.

The adaptive filter according to the present invention can be situated at the digital signals processing part (500) or the vital signals measurement system (800). The adaptive filter described in FIG. 15 is an AR model of FIR structure. While an IIR filter has problems of instability and a slow convergent velocity, an FIR filter has an advantage that the system is stable with an all-pole structure and that local minimum problems do not occur.

It is possible to obtain reliable data by controlling the number of Tab-Delay of a suitable FIR structure according to the level of noise in the adaptive filter. When a start code, a data code, a level of vibration and movement, three axis acceleration values signals of three axis acceleration sensor part, and measured vital signals are provided according to the present invention, an adaptive filter which removes vibration signals included in vital signals can also be modified in various ways by those skilled in the art.

### INDUSTRIAL APPLICABILITY

The present invention relates to a device which displays the vibration index internal to an ambulance on a portable emergency monitoring system by interpreting the vibration signals which effect the physiological state of a body and measurement of vital signals, caused by external disturbance such as vibration inside a moving ambulance, and which compensates for the vibration signal noise during measurement of vital signals. It is possible to apply to most vital signals transmission systems which transmit measured vital signals long distance.

For example, it can be used in systems where ECG is performed in an ambulance or at home and sent to a medical center.

1. In an apparatus for measuring and transmitting vital signals, a system to transmit vital signals from a moving human body in the presence of dynamic external disturbance and to compensate for artifacts thereof, comprising:
a three axis acceleration sensor part detecting each of a X, Y, Z axis acceleration and converting it into electronic signals;
a pre-amplifier part pre-amplifying the output from the three axis acceleration sensor part and supplying only signals within a band for digital signals processing by removing high frequency noise;
an A/D converter converting the output signals from the pre-amplifier part into digital signals;
a digital signals processing part determining stopped or movement conditions of a human being moving on foot or on a vehicle, by mathematically processing the three axis acceleration signals output by the A/D converter; and
a transmitter part transmitting the result of the digital signals processing part and the three axis acceleration signals.

2. The apparatus according to claim 1, wherein the system to transmit vital signals from a moving human body in the presence of dynamic external disturbance and to compensate for artifacts thereof is characterized in that the digital signals processing part can determine whether the human being is moving on foot, has stopped during walking, is being transported by a vehicle which has stopped, or is on a moving vehicle.

3. The apparatus according to claim 1, wherein the system to transmit vital signals from a moving human body in the presence of dynamic external disturbance and to compensate for artifacts thereof is characterized in that the transmitter part transmits measured vital signals together with the result of digital signals processing part and the three axis acceleration signals.

4. The apparatus according to claim 1, wherein the system to transmit vital signals from a moving human body in the presence of dynamic external disturbance and to compensate for artifacts thereof is characterized in that the digital signals processing part comprises:
a memory part storing X, Y, Z axis acceleration data received from the A/D converter in set time frame units;
a differential operation part reading the X, Y, Z axis acceleration data stored in the memory part in set time frame units, and differentiating data signals based on a zero level;
a mean value operation part taking a mean of each of X, Y, Z axis acceleration differential data output by the differential operation part;
a Z axis mean value comparator part determining whether a mean value of Z axis acceleration differential data obtained from the mean value operation part is greater than a first reference value;
a X, Y axis mean value comparator part which compares whether the mean value of X,Y axis acceleration differential data obtained from the mean value operation part is greater than the second reference value, and determines that the vehicle has stopped during moving on a vehicle if it is greater, and determines that the human being has stopped during walking if it is not greater, in case a mean value of Z axis acceleration differential data from the Z axis mean value comparator part is equal to or smaller than the first reference value;
a frequency analysis part obtaining mean spectrum of the Z axis acceleration differential data by calculating FFT of the output signals of the differential operation part; and
a Z axis spectrum comparator part which compare whether the Z axis mean spectrum output from the frequency comparator part is greater than the third reference value and if it is greater determines that the moving human body is moving on foot, and if it is smaller than or equal to the third reference value, determines that the moving body is moving on a vehicle.

5. The apparatus according to claim 1, wherein the system to transmit vital signals from a moving human body in the presence of dynamic external disturbance and to compensate for artifacts thereof characterized in that the digital signals processing part is determines whether the human body is stopped or moving by the mean value of the Z axis acceleration differential signals, and determines whether the human body is stopped during vehicle transport or during travel on foot, by the mean value of the X,Y axis acceleration differential signals.

6. The apparatus according to claim 1, wherein the system to transmit vital signals from a moving human body in the presence of dynamic external disturbance and to compensate for artifacts thereof characterized in that the digital signals processing part determines whether the human body is stopped or moving by a mean value of the Z axis acceleration signals, and determines whether human body is moving on foot or on a vehicle by a spectrum of Z axis acceleration differential signals.

7. The apparatus according to claim 1, wherein the system to transmit vital signals from a moving human body in the presence of dynamic external disturbance and to compensate for artifacts thereof characterized in that the transmitter part transmits the condition data which discriminates stopped and moving states for a human body on foot or on a vehicle output from the digital signals processing part, together with the three axis acceleration data received through the A/D converter from an acceleration sensor (100).

8. A method of transmitting vital signals from a moving human body in the presence of dynamic external disturbance and compensating for artifacts thereof, comprising:
an A/D conversion step converting the signals output from the three axis acceleration sensor part into digital signals, at the A/D converter;
a differential operation step calculating acceleration differential signal against each of the X, Y, Z axis directions, from three axis acceleration signals output by the A/D converter;
a mean operation step obtaining a mean value of the acceleration differential signals against X, Y, Z axis directions by calculating the mean value of the signals output from the differential operation step;
a step determining whether it is a stopped or moving condition by determining from the result of the mean operation step that human body is moving on foot if the mean value of differential signals of Z axis direction is greater than the first reference value, and that a human body has stopped during moving on foot if it is smaller than or equal to the first reference value;
a step determining whether it is a stopped condition during vehicle transport, in case it is determined to be a stopped condition at the step determining whether it is a stopped or moving condition because the mean value of acceleration differential signals of Z axis direction is equal to or smaller than the first reference value, which determines that it is vehicle transport when the mean value of X,Y axis acceleration difference signals is greater than the second reference value and that it is transport on foot if the mean value of X, Y axis acceleration difference signals is smaller than the second reference value;
a spectrum operation step obtaining a mean spectrum of Z axis in case the condition is determined to be moving because the mean value of acceleration differential signals of Z axis direction is greater than the first reference value at the step determining whether it is a stopped or moving condition; and
a spectrum comparing step which determines that the moving is on foot if the mean spectrum value of Z axis is greater than a third reference value, and
determines that the moving is on a vehicle if the mean spectrum of Z axis is smaller than a third reference value by comparing the calculated mean spectrum of Z axis with a third reference value.

9. According to claim 7, the system to transmit vital signals from a moving human body in the presence of dynamic external disturbance and to compensate for artifacts thereof characterized in that
the transmitter part transmits in a packet the condition data discriminating stopped and moving states for a human body on foot or on a vehicle, output from the digital signals processing part, together with the three axis acceleration data, where the data packet begins with a start code (0xFF), followed by data code regarding the current moving condition, then followed by data code regarding the level of vibration and movement, then followed by data code regarding the three axis acceleration values in an X, Y, Z axis sequence.

10. In an apparatus for measuring and transmitting vital signals, the system to transmit vital signals from a moving human body in the presence of dynamic external disturbance and to compensate for artifacts thereof comprising:
a three axis acceleration sensor part detecting each of an X, Y, Z axis accelerations and converting them into electronic signals;
a pre-amplifier part pre-amplifying the output of three axis acceleration sensor part and removing the high frequency noise to supply only signals in the band for digital signals processing;
an A/D converter converting the output signals of the pre-amplifier part into digital signals;
a digital signals processing part operating on the three axis acceleration signals output by the A/D converter and detecting a level of vibration and movement;
a transmitter part transmitting the result of the digital signals processing part, the three axis acceleration signals, and the measured vital signals; and
an adaptive filtering part receiving signals from the transmitter part and removing vibration signals contained in the vital signals.
